(19) United States
(12) Patent Application Publication
(10) Pub. No.: US 2016/0249904 A1
(43) Pub. Date: Sep. 1, 2016

(54) APPARATUS AND METHOD FOR FIXATING A DEVICE TO A BODY TISSUE

(71) Applicant: Yehuda Bachar, Givat Shmuel (IL)

(72) Inventor: Yehuda Bachar, Givat Shmuel (IL)

(21) Appl. No.: 15/035,019

(22) PCT Filed: Nov. 6, 2014
(86) PCT No.: PCT/IL2014/050971

§ 371 Date: May 6, 2016

Related U.S. Application Data
(60) Provisional application No. 61/900,820, filed on Nov. 6, 2013.

(51) Int. Cl. A61B 17/04; A61B 90/00; A61F 2/00; A61B 17/06

(52) U.S. Cl. CPC A61B 17/0401; A61B 17/06; A61B 90/08; A61F 2/0063; A61B 2017/0409; A61B 2017/0464; A61B 2017/06052; A61B 2090/08021; A61F 2002/0072

(57) ABSTRACT

The subject matter discloses an apparatus for fixating anchors to a body tissue, comprising a housing, two or more interconnected anchors mounted inside the housing and configured to be reinforced to the body tissue, wherein the housing has a first position in which the housing forms an aperture that enables release of an anchor of the two or more interconnected anchors and a second position preventing release of an anchor of the two or more interconnected anchors. The apparatus may be used to close openings in the abdominal cavity or to reinforce a mesh to a body tissue. The anchors released from the apparatus may be connected by filaments.

### FIELD OF THE INVENTION

The invention generally relates to fixation of reinforcing materials or elements to the undersurface of a body wall.

### BACKGROUND OF THE INVENTION

In some surgical operations, surgical supports, for example, meshes, are employed by surgeons to bridge, repair and/or reinforce tissue defects within a patient, especially those occurring in the abdominal wall, chest wall, diaphragm and other areas of the body. During such operations, surgeons employ conventional or known suturing or anchoring techniques to apply such supports to body tissue. For example, U.S. Pat. Nos. 4,652,245 and 5,203,864 describe methods for suturing or anchoring mesh supports to body tissue, especially during hernia repair operations.

In the last decade or so, a minimally invasive surgical approach to ventral hernia repair has become possible and more commonly used. In this approach, several laparoscopic instruments are introduced through small incisions into the abdomen. The instruments include a visualization scope, a pressurization pathway to insufflate the abdomen, and two or more manipulating instruments (e.g., grasping forceps, staplers, tackers, suturing devices, etc.). The instruments are manipulated to introduce and place the mesh under the hernia defect with a wide margin of mesh outside the defect. The mesh is anchored into place with suture and/or secured to the anterior abdominal wall with a varying number of tacks, placed laparoscopically. The mesh minimizes tension on the repair. The instruments are removed and the several wounds extending into the abdominal cavity are closed.

### SUMMARY OF THE INVENTION

It is an object of the subject matter to disclose an apparatus for fixating anchors to a body tissue, comprising a housing and two or more interconnected anchors mounted inside the housing and configured to be reinforced to the body tissue. The housing has a first position in which the housing forms an aperture that enables release of an anchor of the two or more interconnected anchors and a second position preventing release of an anchor of the two or more interconnected anchors.

In some cases, the housing comprises a retractable needle located in a distal end of the housing.

In some cases, the needle protrudes from the two or more interconnected anchors when the housing is in the second position. In some cases, an anchor of the two or more interconnected anchors is released from the apparatus after the needle enters the body tissue. In some cases, the housing has a third position in which the needle does not protrude from the two or more interconnected anchors while preventing release of an anchor of the two or more interconnected anchors.

In some cases, the two or more interconnected anchors are connected via filaments. In some cases, at least a portion of the filaments are wrapped around the two or more interconnected anchors before released from the apparatus. In some cases, a portion of the filaments are kept outside the body tissue when the anchors are released in the body tissue. In some cases, the two or more interconnected anchors have variable cross-sectional area, wherein the filaments are wrapped on a narrow portion of the two or more interconnected anchors.

In some cases, the apparatus further comprises a cradle for storing the anchors, said cradle is located inside the housing and has a distal blunt tip. In some cases, an inner wall located at a distal end of the cradle forms a slope between the other anchors positioned in the cradle during release of an anchor from the aperture.

In some cases, the apparatus further comprises a pusher for pushing the two or more interconnected anchors when releasing the anchor from the aperture. In some cases, the pusher comprises a ratchet mechanism. In some cases, the pusher is controlled from a proximal end of the apparatus.

In some cases, the apparatus further comprises a retracting mechanism for retracting the housing after release of an anchor from the apparatus. In some cases, the needle is provided with a cutting element for cutting a filament connected to an anchor. In some cases, the needle is hollow and surrounds the elongated body.

It is another object of the subject matter to disclose an apparatus, comprising a housing comprising a retractable needle located in a distal end of the housing and two or more anchors mounted in a cradle having a blunt tip, said cradle is inside the housing. The blunt tip may be retracted and the sharp needle exposed during penetration through a more resilient tissue.

In some cases, an anchor of the two or more anchors is released from the housing when the cradle protrudes from the needle.

In some cases, the apparatus further comprises a spring connected to the cradle, wherein the spring is compressed and the cradle is retracted when the resilient tissue is hard, enabling the needle to protrude beyond the cradle.

It is another object of the subject matter to disclose an apparatus comprising a first anchor and a second anchor connected by a filament, wherein the filament is firmly attached to the first anchor and a second anchor; wherein the filament's length is significantly longer then the distance between an attachment point of the first anchor and an attachment point of the second anchor, wherein the filament is tightly packaged in relation to the first anchor and the second anchor when the first anchor and the second anchor are axially stored within a delivery system of the apparatus.

In some cases, the first anchor comprises a receptacle portion for receiving a protruding portion of the second anchor, a protruding portion configured to be mounted on a receptacle portion of a third element and a narrow portion on which the filament is wrapped.

In some cases, the third element is chosen from a group of: a pusher, or an anchor. In some cases, the protruding portion is a ball-like shape. In some cases, the anchor further comprises a fixation mechanism for fixating a filament connecting the first anchor and the second anchor, said fixation mechanism prevents the anchor from sliding on the filament.

It is another object of the subject matter to disclose a method of assembling an apparatus, said method comprises interconnecting two anchors connected by a filament using a ball and socket connection; holding the two anchors at an angle other than 1800;rotating a first anchor relative to a second anchor of the two anchors and wrapping the filament on the first anchor; aligning the two anchors using mounting noncircular connecting components correspondingly to prevent relative axial rotation between the first anchor and the second anchor.

In some cases, the method further comprises introducing the two connected anchors into a housing cradle of the apparatus.

It is another object of the subject matter to disclose a method for attaching a mesh to a cavity wall, comprising introducing a mesh into the cavity; penetrating with an apparatus comprising anchors through the cavity wall; approximating the mesh to a body tissue; penetrating with the apparatus comprising anchors connected by filaments through the mesh at a first location; penetrating with the apparatus comprising anchors through the body tissue; releasing a first anchor from the apparatus within the body tissue; retracting the apparatus from the body tissue and from the mesh within the cavity wall; penetrating with the apparatus comprising anchors connected by filaments through the mesh at a second location; penetrating with the apparatus comprising anchors through the body tissue at a second location; releasing a second anchor within the body tissue at the second location; retracting the apparatus from the body tissue and from the mesh within the cavity wall; said second anchor being connected to the first anchor via a filament.

In some cases, the method further comprises inflating the cavity before penetrating to the cavity wall. In some cases, the apparatus is inserted percutaneously through the abdominal wall without the need for a trocar. In some cases, the apparatus comprises a retractable needle. In some cases, the cavity is the abdominal cavity. In some cases, the body tissue is a fascia. In some cases, the method further comprises cutting the filament. In some cases, the method further comprises removing the apparatus from the cavity.

It is another object of the subject matter to disclose a method for closing openings in an abdominal wall, comprising introducing an apparatus comprising an anchor connected to a filament through an opening, said apparatus is inserted facing towards the abdominal cavity; penetrating a fascia near the opening margins; releasing a first anchor connected to the first filament below the fascia; retracting the apparatus out of the opening; penetrating the fascia with an apparatus comprising an anchor connected to a filament near the opening margins at a second location; releasing a second anchor connected to a second filament below the fascia at a second location; retracting the apparatus out of the opening.

In some cases, the method further comprises knotting a first filament connected to the first anchor and the second filament connected to the second anchor. In some cases, the apparatus comprises a retractable needle.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A shows the housing and anchors of the apparatus, in accordance with a preferred embodiment of the invention;

FIG. 1B schematically shows the housing, cradle and anchors of the apparatus, in accordance with a preferred embodiment of the invention;

FIG. 2 shows the pusher mechanism and retracting mechanism of the apparatus, in accordance with a preferred embodiment of the invention;

FIG. 3 shows the apparatus in a needle front position, in accordance with a preferred embodiment of the invention;

FIG. 4 shows the apparatus in a position that enables release of an anchor, in accordance with a preferred embodiment of the invention;

FIG. 5 shows the apparatus in a blunt tip and release prevention position, in accordance with a preferred embodiment of the invention;

FIG. 6 shows the apparatus penetrating the body tissue, in accordance with a preferred embodiment of the invention;

FIG. 7 shows the apparatus releasing a first anchor into the body tissue, in accordance with a preferred embodiment of the invention;

FIG. 8 shows the apparatus releasing two connected anchors into the body tissue, in accordance with a preferred embodiment of the invention;

FIG. 9 shows a method of reinforcing a body tissue, in accordance with a preferred embodiment of the invention;

FIGS. 10A-10B show the handle of the apparatus being connected to the elements inserted into the body tissue, in accordance with a preferred embodiment of the invention;

FIGS. 11A-11D show the anchor of the apparatus from various angles, in accordance with a preferred embodiment of the invention;

FIGS. 12A-12D show a pair of interconnected anchors from various angles, in accordance with a preferred embodiment of the invention;

FIGS. 13A-13B show an anchor released from the subsequent anchor, in accordance with a preferred embodiment of the invention;

FIG. 14 shows a method of closing openings in an abdominal wall, according to exemplary embodiments of the disclosed subject matter;

FIGS. 15A-15D show an apparatus for closing openings in an abdominal wall, according to exemplary embodiments of the subject matter; and,

FIG. 16 shows a method of assembling an apparatus, according to exemplary embodiments of the disclosed subject matter.

### DETAILED DESCRIPTION OF THE INVENTION

The present invention discloses an apparatus for fixating anchors to a body tissue. The apparatus is inserted into the body from a wall and penetrates the body tissue from the internal tissues outwards. The anchors are used to close an opening in the wall of the body tissue, and/or to attach a mesh to the body tissue in order to reinforce the body tissue, for example hernia regions of the abdominal wall or weakened regions of abdominal wall after abdominal surgeries. The apparatus uses a sharpened element such as a needle to penetrate to the body tissue in which the anchors are configured to be released. The needle can be an integral part of the housing of the apparatus or can be attached to the apparatus. The anchors may be stored in a cradle inside the housing, connected to a pusher mechanism controlled by a user of the apparatus which maneuvers the apparatus and decides where and when to release the anchors.

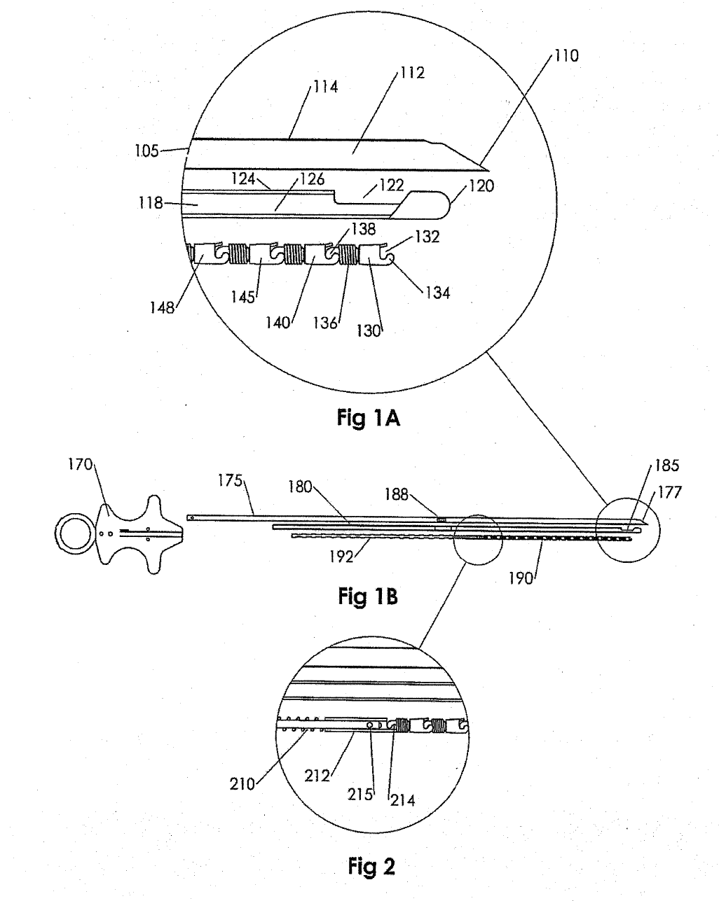

FIG. 1A shows the components of the housing, cradle and anchors of the apparatus, in accordance with a preferred embodiment of the invention. The housing 105 of the apparatus is configured to store a plurality of anchors used to reinforce a material or a sheet to a body tissue. The housing 105 may be equipped with a sharpened needle 110 in its distal end. The housing 105 has a hollow volume 112 in which the plurality of anchors are stored. The plurality of anchors may be stored in a cradle 118 located in the hollow volume 112 of the housing 105. The walls 114 of the housing 105 may be of a cylindrical cross-sectional shape.

The cradle 118 has an aperture 122 via which the plurality of anchors are deployed from the apparatus. The aperture 122 is a recess in the wall 124 of the cradle 118. In some exemplary cases, the anchors can be released via the aperture 122 according to the position of the housing 105 versus the cradle 118. In some other cases, an anchor of the plurality of anchors 130, 140, 145, 148 is released via another mechanism, without a cradle, as the anchors are in contact with the inner walls of the housing 105. In some exemplary cases, the plurality of anchors 130, 140, 145, 148 are contained in a hollow volume 126 of the cradle 118. The cradle 118 may be equipped with a blunt tip 120, which protrudes beyond the needle 110 when the needle 110 is retracted by the user of the apparatus, as detailed below.

The plurality of anchors 130, 140, 145, 148 are configured to be released in a predefined body tissue, or in a specific layer of the body tissue. The plurality of anchors 130, 140, 145, 148 may be interconnected, for example via filaments 136. Such filament 136 may be a suture material manufactured of material such as: nylon, polyester, polypropylene, a biodegradable material such as PLA, PLGA, POLYCAPROLACTONE, etc. The plurality of anchors 130, 140, 145, 148 may be manufactured of similar plastic materials or of metal such as: stainless steel, titanium, nitinol, etc. The filament 136 may be wrapped around the anchor 130 when kept inside the apparatus. Then, when the anchors are released from the apparatus, the turns of the wrapping of the filament 136 are released from the anchor 130, permitting the filament 136 to straighten. The length of the plurality of anchors 130, 140, 145, 148 may be between 3 to 15 mm and in particular embodiments between 5 to 9 mm. The length of the connecting filament may be 25 to 40 mm and in a specific embodiment 30 to 35 mm. Wrapping the filament 136 on the plurality of anchors 130, 140, 145, 148 prevent any slack filament that may block the advancement of the plurality of anchors 130, 140, 145, 148. The needle 110 may be used to cut the filaments according to the user's needs, for example after release of two anchors, to enable release of another pair of anchors at another location. Deployment the anchors may be done continuously, for example by releasing all the anchors on one batch, as all the anchors are connected in series resulting in a continuous attachment of the mesh to the tissue. The attachment of the reinforcing mesh may be discrete, as each time a pair of anchors are released, to attach the mesh at a specific spot in the body tissue.

Before being released from the housing 105, the plurality of anchors 130, 140, 145, 148 may be connected to each other via a “male-female” connector, in which every anchor has a protruding part and a receptacle part. For example, anchor 130 has a receptacle part 132 and a protruding part 138 received and held by a receptacle part of anchor 140. The receptacle part 132 forms a volume 134 for receiving a protruding part of another anchor to be interconnected to the anchor 130.

In some exemplary cases, the apparatus has two positions. In the first position, an anchor of the plurality of anchors 130, 140, 145, 148 can be released from the apparatus according to the position of the housing 105 relative to the cradle 118. In the first position, the distal end of the cradle 118, optionally formed as a blunt tip 120, protrudes beyond the housing 105, which results in the aperture 122 being in contact with the body tissue, such that an anchor can be released to the body tissue. In the second position, the distal end of the housing 105 protrudes beyond the blunt tip 120, for example when the needle 110 penetrates into the body tissue. The apparatus may have a third position, in which the blunt pin 120 protrudes beyond the needle 110 and the aperture 122 is at least partially blocked by the walls 114 of the housing 105.

FIG. 1B schematically shows the housing, cradle and anchors of the apparatus, in accordance with a preferred embodiment of the invention. The apparatus is operated by a handle 170 controlled by a user of the apparatus, for example a medical doctor, a surgeon, a medic and the like. The handle 170 may be operated automatically by a robot. The handle 170 may comprise an input unit that enables the user of the apparatus to input commands or information into an electronic module of the apparatus, for example a command may be to move forward as long as the doctor presses a button, as the button represents the input unit.

The handle 170 is connected to the plurality of anchors 190 via a pusher mechanism 192. The plurality of anchors 190 and the pusher mechanism 192 are located in the cradle 180 and the anchors 190 are released from the cradle 180 via the aperture 185 when the needle 177 is retracted back from the aperture 185. The cradle 180 has a stopper 188 which limits the movement of the plurality of anchors 190 when retracted after releasing an anchor via the aperture 185. The cradle 180 is located inside a housing 175, for example having a cylindrical cross sectional shape. The plurality of anchors 190 may be contained directly in the housing 175 without the need of the cradle, and released upon a command from the input unit of the handle 170. The pusher mechanism 192 is controlled by the user of the apparatus, for example via the handle 170. The pusher mechanism 192 may be implemented as a ratchet mechanism, in which every time the user of the apparatus advances the pusher mechanism 192 a single step, one anchor is pushed from the apparatus via the aperture.

FIG. 2 shows the pusher mechanism of the apparatus, in accordance with a preferred embodiment of the invention. A spring 210 is connected to the pusher mechanism 192 to enable the plurality of anchors to be pushed against the stopper at the tip of the cradle and the cradle tip is biased to protrude beyond the tip of the hollow outer needle.

Recession of the blunt tip of the cradle by the pressure exercised by the tissue together with the plurality of anchors enables the protrusion of the outer sharpened needle in order to penetrate the body tissue. A connector 212 connects the spring 210 to the first anchor 214 of the plurality of anchors. The first anchor 214 is located at the proximal side of the plurality of anchors, as the plurality of anchors reside in the cradle. The distal tip of the cradle is a blunt tip, while the distal end of the housing surrounding the cradle is a sharpened needle. When the apparatus faces a soft tissue, the blunt pin protrudes from the needle and prevents any damage in a body tissue. When the tissue is hard, the spring 210 compresses, the blunt pin is retracted and the needle protrudes and comes in contact with the tissue. The mechanism of recession of the blunt pin according to the tissue strength via a spring or another elastic mechanism may be used in non-medical methods in which there is a need to penetrate into a tissue having various strengths.

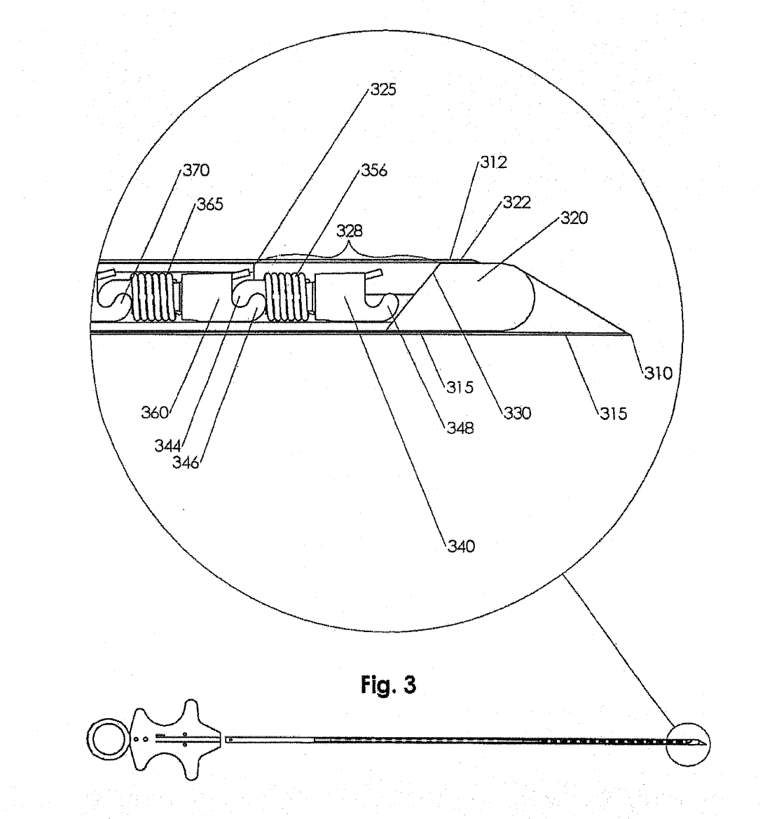

FIG. 3 shows the apparatus in a needle front position, in accordance with a preferred embodiment of the invention. FIG. 3 shows a position of the apparatus in which the needle 310 of the housing 315 protrudes beyond the blunt tip 320 of the cradle 322. This needle front position is likely to be used when penetrating into a body tissue, for example when inserting the apparatus into the patient's body, or when inserting the apparatus into the body tissue in which the anchors are released. The anchors 340, 360, 370 located inside the cradle 322 are released when pushed by the pusher mechanism via the aperture 328. The aperture 328 is defined as the area between distal inner wall 330 of the cradle 322 and the distal end 325 of the cradle cover. In the needle front position, a wall 312 of the housing 315 covers the aperture 328 and prevents the anchor 340 from being released to the body tissue.

When pushed, the most distanced anchor, for example anchor 340, is slid on the distal inner wall 330 of the cradle 322. The distal inner wall 330 forms a slope relative to the inner surface of the cradle 322, and facilitates the deployment of the anchors via the aperture 328. A filament 356 is wrapped around the narrow portion of the anchor 340. The filament 356 wrappings are released from the anchor 340 after the anchor 340 is released from the apparatus, and the anchor remains connected to the filament 356 at the end of the filament. The filament 356 connects one anchor to the other, for example connects anchor 340 to anchor 360 after they are released at the body tissue. The anchor 340 has a receptacle portion 348 which facilitates the binding of another anchor thereto, when the anchors are stored in the cradle 322. The anchor 340 has a protruding portion 344 mounted on a receptacle portion 346 of anchor 360. Similarly, anchor 360 is mounted in a receptacle portion of the anchor 370. Filament 365 connects the anchors 360 and 370 after being released from the apparatus. The connection of the anchors 360, 370 to the filament 365 may be a firm connection preventing any sliding of the anchor on the filament, or the connection to the filament may be by an engagement of the filament 365 that permit the anchor 360 to glide in one direction or in both directions. In some exemplary cases, only some of the anchors in the cradle 322 are connected to each other. For example, the first anchor is connected to the second anchor in line and the second anchor is not connected to the third anchor. The third anchor is connected to the fourth anchor, such that each time, only two anchors are connected to each other.

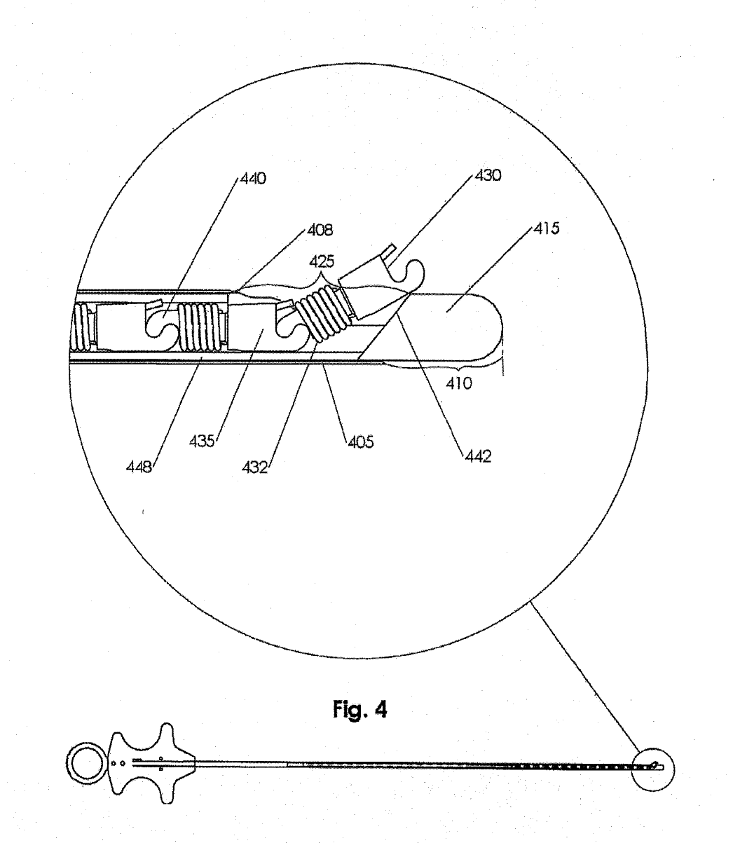

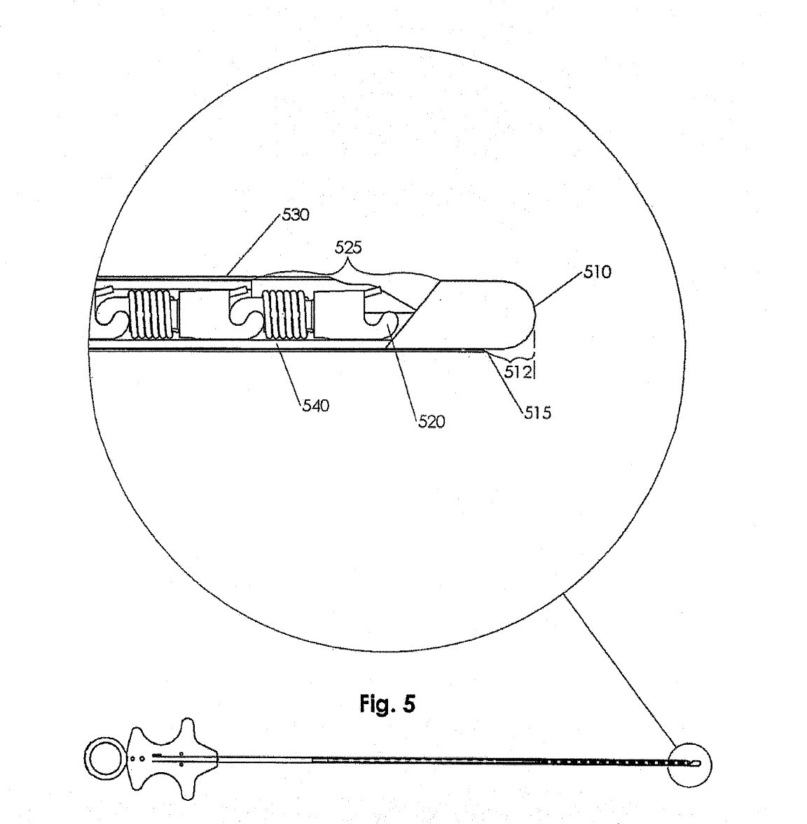

FIG. 4 shows the apparatus in a position that enables release of an anchor, in accordance with a preferred embodiment of the invention. In FIG. 4, the distal end of the housing 405 is distanced from the blunt tip 415 of the cradle. The distance 410 between the blunt tip 415 and the distal end of the housing 405 and the distance between cover 408 of the aperture 425 and the distal inner wall 442 of the cradle indicate that the anchor 430 can be released from the cradle when required by the user of the apparatus. The aperture 425 is open and enables the anchor 430 to pass there through. The anchor 430 is in direct contact with the anchor 440 before being released from the cradle to the body tissue. After being secured in the body tissue, the anchor 430 is connected to anchor 440 via filaments 432. When released from the housing via the aperture 425, an angle is formed between the position of the anchor 430 and the inner surface 448 of the cradle. The length of the aperture may be 2-10 mm, or more specifically 5 to 8 mm.

The released anchor 430 is pushed by the anchor 440 connected thereto via the receptacle element 435 disclosed above. The anchor 440 is pushed by other anchors located in the cradle towards the pusher mechanism operated by the user of the apparatus. After the anchor 430 is released from the aperture 425, the cradle is retracted back using the handle, to prevent additional anchors to be deployed from the apparatus via the aperture 425

FIG. 5 shows the apparatus in a blunt tip and release prevention position, in accordance with a preferred embodiment of the invention. In the third position disclosed herein, the blunt tip 510 protrudes beyond the needle 515 and the aperture 525 of the cradle is partially covered by the wall 530 of the housing, preventing the anchor 520 from being released to the body tissue. The anchor 520 is mounted on an inner surface 540 of the cradle. In such neutral position, the needle 515 is minimally retracted, and the blunt tip 510 of the cradle protrudes, to prevent injury to adjacent tissues. In this configuration, the lateral window is partially open and cannot permit releasing the distal anchor 520. This will prevent the anchor 520 from being released after a previous anchor is released, when there is no need to penetrate into a body tissue and there is no need to deploy another anchor. The third position may be defined by the lateral distance 512 between the needle 515 and the blunt tip 510. For example, the lateral distance 512 is smaller than a predefined length, such as 3 mm, in the third position, and 7 mm in the second position, in which the anchor can be released via the aperture 525.

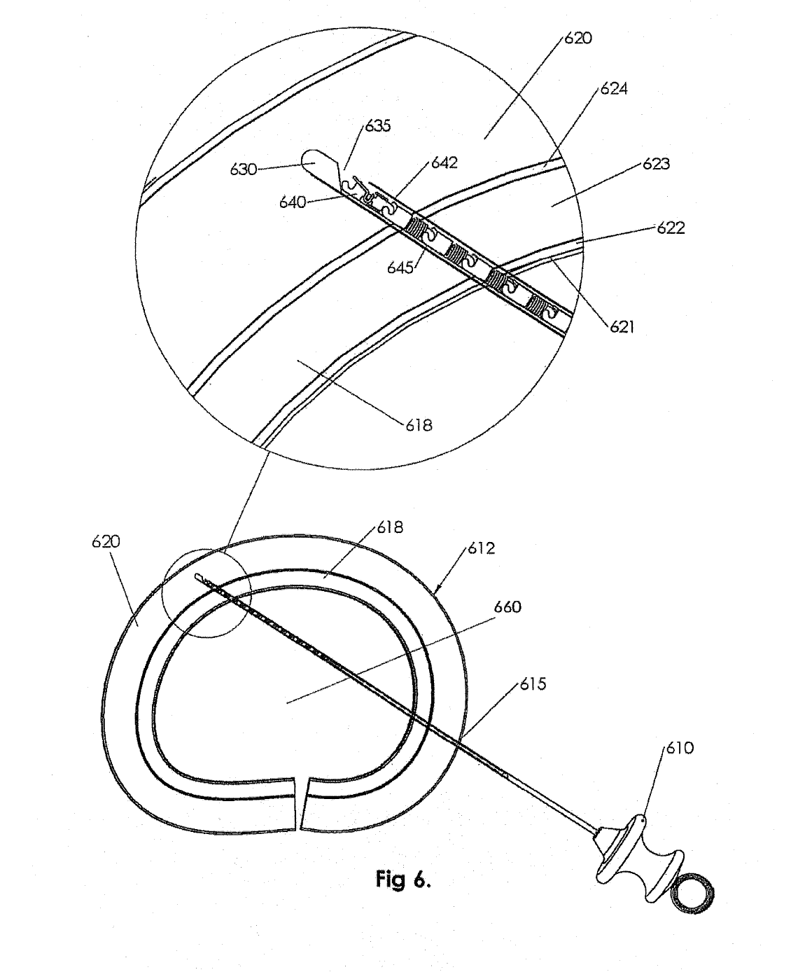

FIG. 6 shows the apparatus penetrating the body tissue, in accordance with a preferred embodiment of the invention. The user of the apparatus controls the apparatus via the handle 610 and inserts the apparatus into the patient's body via an entry point 615 on the patient's external body tissue 612, such as a skin. During laparoscopy, the abdominal cavity is inflated with gas. The apparatus passes through the abdominal wall into the abdominal cavity. Thereafter, the apparatus is directed under laparoscopic vision to the site where the mesh has to be attached to the body wall. The device is advanced through the mesh 621 and the peritoneum 622, fatty tissue 623 and fascia 624. For example, the apparatus passes through the internal body tissues 622, 623 and 624 before penetrating the body tissue 620, where the anchor will be deployed for example using the needle. When the apparatus passes from the body tissue 624 to the body tissue 620, the user pushes an anchor via the pushing mechanism, such that the anchor 640 is released via the aperture 635 into the body tissue 620. While the anchor 640 is released, the blunt tip 630 protrudes over the needle. The handle 610 may comprise an indication unit to indicate whether the needle protrudes over the blunt tip 630 or the opposite. The anchors are kept in a housing or in a cradle defined by inner walls 642, 645. After releasing the anchor 640, the apparatus is moved back to the abdominal cavity 660, while tensioning the filament connecting the released anchor to the next anchor within the device, and the user of the apparatus moves the apparatus to the next point of penetration to the body tissue 620 and the sequence is repeated. This results in the anchors being released above the fascia. Only the connecting filaments will be exposed on the undersurface of the reinforcing mesh. This results in firm attachment of the mesh to the reinforced tissue. It should be noted that multiple penetrations to the body tissue 620 are made from the internal layer of body tissue 618 outwards, to firmly attach the mesh to the abdominal wall.

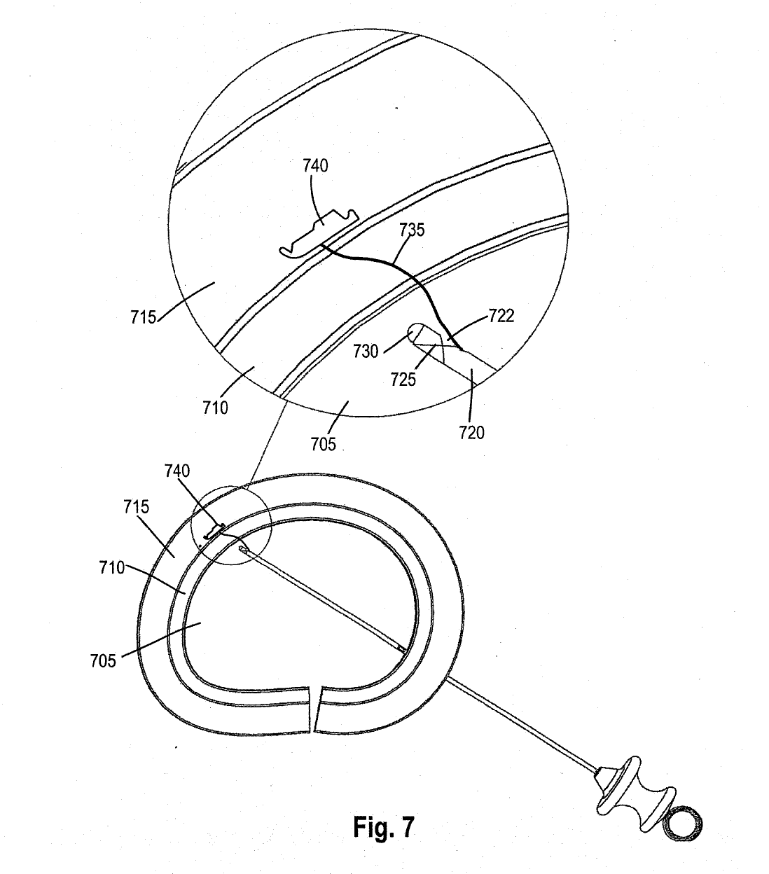

FIG. 7 shows the apparatus releasing a first anchor into the body tissue, in accordance with a preferred embodiment of the invention. The first anchor 740 is released into the reinforced body tissue 715 via an aperture 722 created when the housing 720 is in a specific position relative to the cradle having the blunt tip 730. The needle 725 is used when the apparatus has to penetrate a body tissue, for example when penetrating from intermediate body tissue 710 to reinforced body tissue 715. After releasing the anchor 740 at the reinforced body tissue 715, the apparatus is retracted by the user to the abdominal cavity 705 where the apparatus is moved to find the next reinforcing point as shown in FIG. 8. The first anchor 740 is connected to the next anchor via filaments 735.

FIG. 8 shows the apparatus after releasing two connected anchors into the body tissue, in accordance with a preferred embodiment of the invention. After the first anchor 840 is released into the reinforced body tissue 815, the apparatus 800 is retracted by the user back to the abdominal cavity tissue 805 until the user finds the proper location for releasing the second anchor 825 in the reinforced body tissue 815. Before releasing the second anchor 825, the apparatus 800 is required to penetrate the mesh and reinforced tissue from within body tissue 810 and into reinforced body tissue 815. The first anchor 840 and the second anchor 825 are connected by a filament 845. The anchors and the filament attach firmly the mesh to the body tissue at this location. The user of the apparatus 800 may cut the filament on the body tissue after releasing the second anchor 825. In some cases, the same filament can connect more than two anchors released in the reinforced body tissue 815.

In some exemplary cases, the reinforced body tissue may be the abdominal cavity 805 and the location may be a weakness or hernia of the cavity wall. Such hernia may be any one of these but not limitedly: an inguinal hernia; a postoperative ventral hernia, a femoral hernia, lumbar hernia, other anterior abdominal wall hernia, diaphragmatic hernia, parastomal hernia, pelvic organ floor related hernia or weakness (rectal prolapse, vaginal prolapse, uterine prolapse), etc.

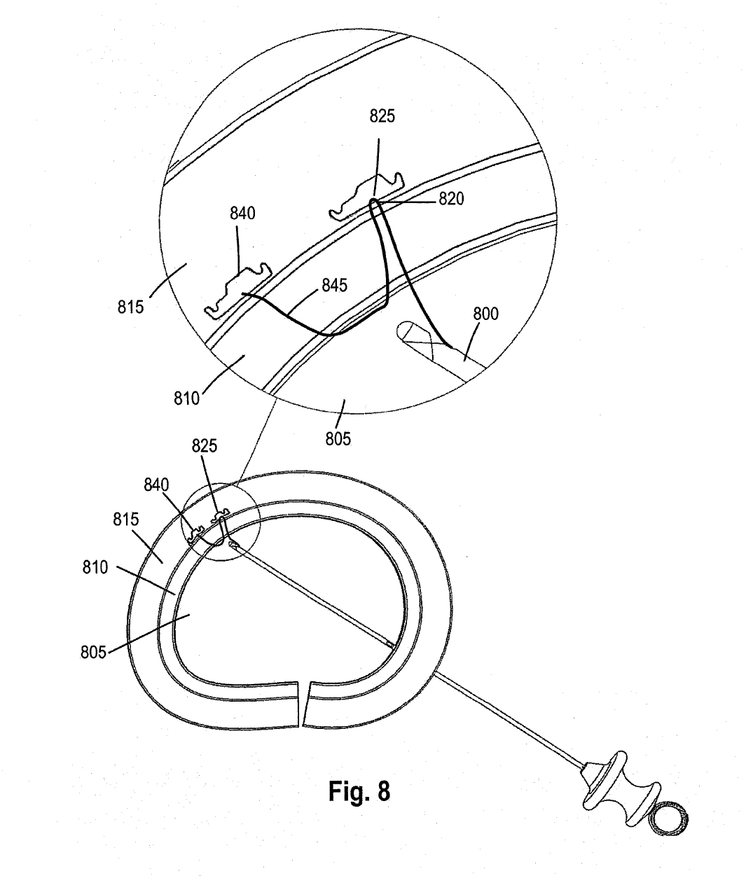

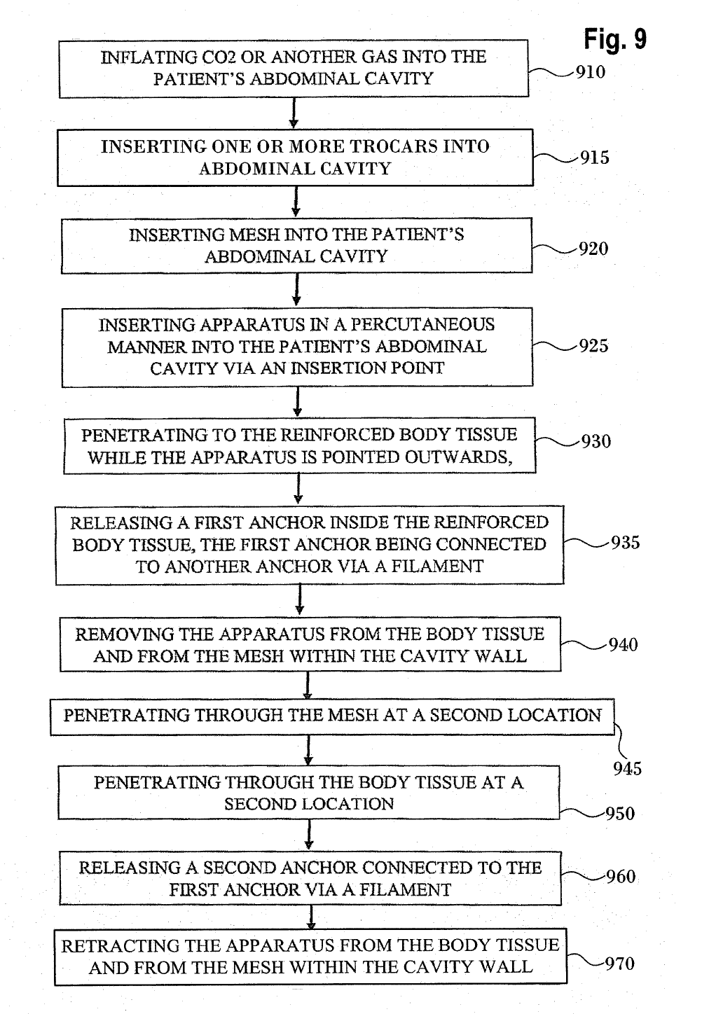

FIG. 9 shows a method for reinforcing a mesh of material to a cavity wall, in accordance with a preferred embodiment of the invention. Step 910 discloses inflating CO2 or another gas into the patient's abdominal cavity. The amount of CO2 injected therein may be determined by the doctor during operation. In step 915, one or more trocars are inserted into the abdominal cavity. In some cases, at least one of the trocars comprises an endoscope, or another device carrying a camera. In step 920, the mesh is inserted into the patient's abdominal cavity, for example via one of the trocars, and attached to the body tissue that is to be reinforced, as defined above. Then, in step 925, the apparatus is inserted in a percutaneous manner into the patient's abdominal cavity via an insertion point. Step 930 discloses the apparatus penetrating to the reinforced body tissue while the apparatus is pointed outwards, towards the patient's skin. Step 935 discloses releasing a first anchor inside the reinforced body tissue, the first anchor being connected to another anchor via a filament. The first anchor is released above the fascia tissue. Step 940 discloses removing the apparatus from the body tissue and from the mesh within the cavity wall. The apparatus is be retracted back to the abdominal cavity. Step 945 discloses the apparatus penetrating through the mesh at a second location. Step 950 discloses the apparatus penetrating through the body tissue at a second location. The apparatus penetrate into two different points in the mesh attached to the tissue. Step 960 discloses releasing a second anchor connected to the first anchor via a filament. The filament is located, at least partially, outside the reinforced body tissue and passes bellow the mesh attaching the mesh firmly to the abdominal wall. Step 970 discloses retracting the apparatus from the body tissue and from the mesh within the cavity wall.

These steps may be repeated as needed resulting in a continuous attachment of the mesh to the abdominal wall. Or alternatively, the filament connecting the second deployed anchor to the apparatus may be cut resulting in a discrete attachment of the mesh to the body tissue. The filament may be cut by the apparatus by other laparoscopic scissors.

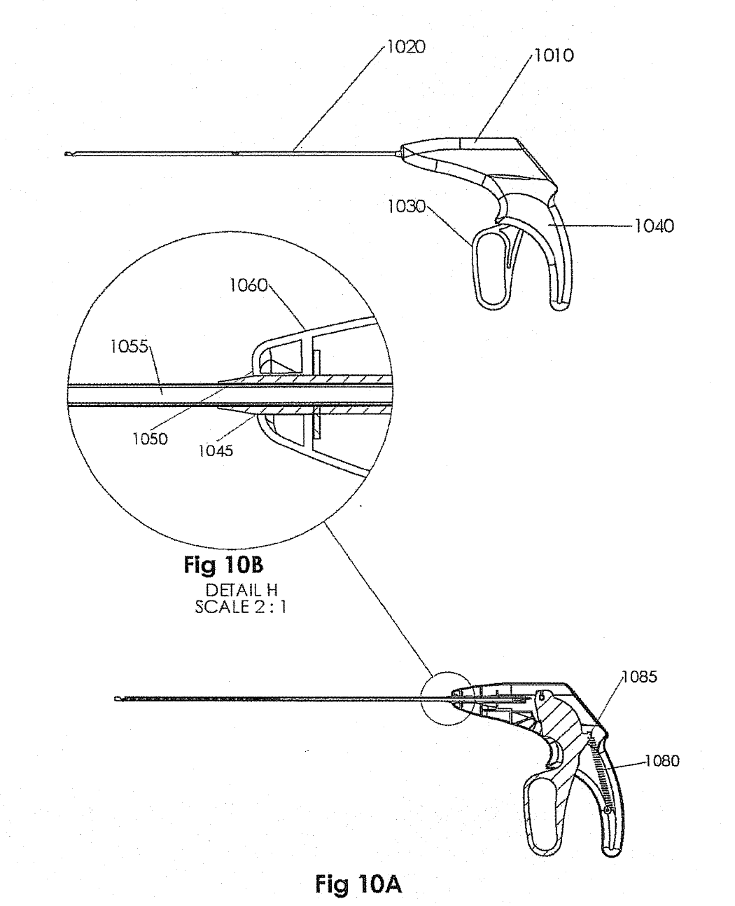

FIGS. 10A-10B show the handle of the apparatus being connected to the elements that are inserted into the body tissue, in accordance with a preferred embodiment of the invention. The housing 1020 is connected to a body 1010 of the apparatus at connection point located outside the body 1010. The body 1010 is connected to the handle 1030 used by the user to operate the apparatus and to release an anchor into a tissue, such as a body tissue. The housing 1020 is affixed to the body 1010, in a manner that the distance between the distal end of the housing that comprises a needle is constant during operation of the apparatus. The rear portion 1040 of the body 1010 is used to balance the user's movement of the handle 1030.

FIG. 10B shows the cradle 1055 connected to the upper portion 1050 of the handle 1030. The handle moves around a hinge (not shown), such that when the user pulls the handle 1030 backwards, the upper portion 1050 moves forward and pushes the cradle 1055 forward. A spring 1080 is compressed when the handle 1030 rests in front position, and tensed when the handle 1030 is pulled backwards by the user. The handle 1030 and the spring 1080 may be connected in connection point 1085 at the upper portion of the spring 1080. The cradle 1055 is movable inside the housing 1020 responsive to the upper portion 1050 of the handle 1030 pushing the proximal portion 1045 of the cradle 1055. The cradle 1055 slides inside the housing 1010 below the upper front portion 1060 of the body 1010.

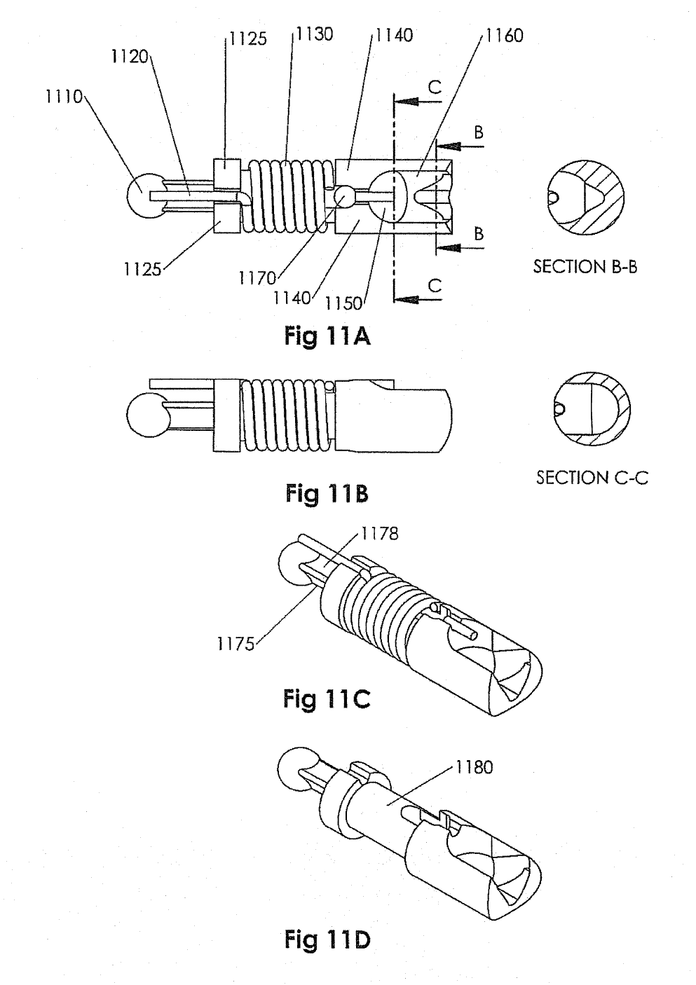

FIGS. 11A-11D show the anchor of the apparatus from various angles, in accordance with a preferred embodiment of the invention. The anchor comprises a protruding portion 1110 configured to be inserted into a receptacle of another anchor, for example a second anchor positioned next to the first anchor in a cradle of the apparatus. The protruding portion 1110 may have a ball-like shape, to facilitate wrapping of a filament 1130 around a narrow portion 1180 of the anchor when inserting the anchors to the cradle. The protruding portion 1110 may be connected to a narrow neck 1120 located between the protruding portion 1110 and the first body part 1125 of the anchor. The narrow neck 1120 may be used to facilitate the protruding portion 1110 of the anchor to move upwards before released from the housing while the first body part 1125 of the anchor is still inside the cradle. The first body part 1125 is connected to the narrow portion 1180, and keeps the filament 1130 around the narrow portion 1180 only, such that the cross section of the anchor is not extended due to wrapping the filament 1130 around the anchor.

The narrow portion 1180 may be connected to a second body portion 1140 having a receptacle 1160 for receiving a protruding portion of a third anchor positioned next to the receptacle 1160. The receptacle 1160 may be designed as a triangular cross section having a narrow end 1175 in a bottom portion and a wide end 1178 in the upper portion, to prevent the third anchor from being rotated while mounted in the receptacle 1160. The receptacle 1160 may be of another shape desired by a person skilled in the art to prevent such rotation. The receptacle 1160 may have a deeper segment 1150 designed as a circle to enable insertion of a protruding portion designed as a ball, similar to protruding portion 1110.

The second body portion 1140 may be connected to a stopper 1170 configured to fix the filament and limit the sliding of the anchor over the filament 1130 after the anchor is released from the apparatus. In such a case, the distance between the anchor and the next anchor is constant after they are released, determined by the length of the filament wrapped around the anchor. In some other cases, the anchor does not include a stopper 1170 and the anchor can slide on the filament 1130.

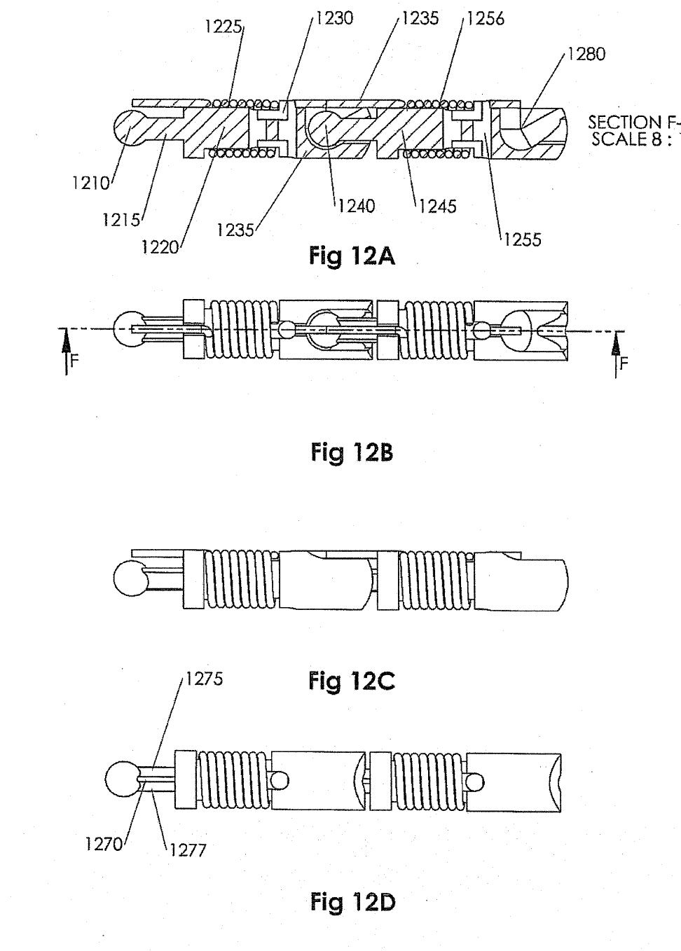

FIGS. 12A-12D show a pair of interconnected anchors from various angles, in accordance with a preferred embodiment of the invention. The first anchor and the second anchor are connected via a protruding portion 1240 of the second anchor inserted into a receptacle 1235 of the first anchor. The first anchor comprises a protruding portion 1210 connected to a narrow neck 1215 as described above. The narrow neck 1215 is connected to a first body portion and a narrow portion 1220 having a filament 1225 wrapped around the narrow portion 1220. The narrow portion 1220 is connected to the receptacle 1235 via a second body portion. The protruding portion 1240 of the second anchor is connected to a narrow neck connected to a body portion of the second anchor. The body portion is connected to a narrow portion 1245 having a filament 1250 wrapped around. The filament 1250 is limited by a second body portion 1255 connected to the receptacle 1280 of the second anchor. In some exemplary cases, the receptacles 1235, 1280 of the anchors are shaped in a way that prevents a protruding element of another anchor from rotating inside the receptacle. Such shape may be polygonal, for example a triangle having a narrow end 1270 and two sides 1275 and 1277 forming a slope from the narrow end to have a wide end in the upper portion of the receptacle 1280.

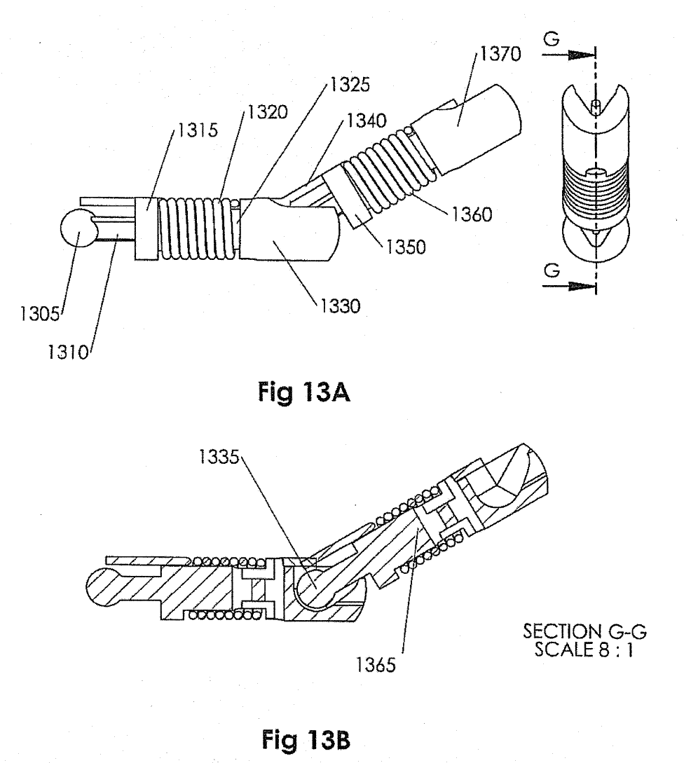

FIGS. 13A-13B show an anchor released from the subsequent anchor, in accordance with a preferred embodiment of the invention. The protruding element 1305 of the first anchor is connected to a narrow neck 1310. The narrow neck 1310 is connected to a first body portion 1315 connected to a narrow element 1325 having a filament 1320 wrapped around. The narrow element 1325 is connected to a receptacle 1330 into which a second protruding element 1335 of the second anchor is inserted. The second protruding element 1335 may be of a ball-like shape to enable rotational movement of the second anchor when released from the receptacle 1330, and hence from the apparatus. The second protruding element 1335 of the second anchor is connected to a narrow neck 1340. The narrow neck 1340 is connected to a first body portion 1350 connected to a narrow element 1365 having a filament 1360 wrapped around. The narrow portion is connected to a receptacle 1370 of the second anchor.

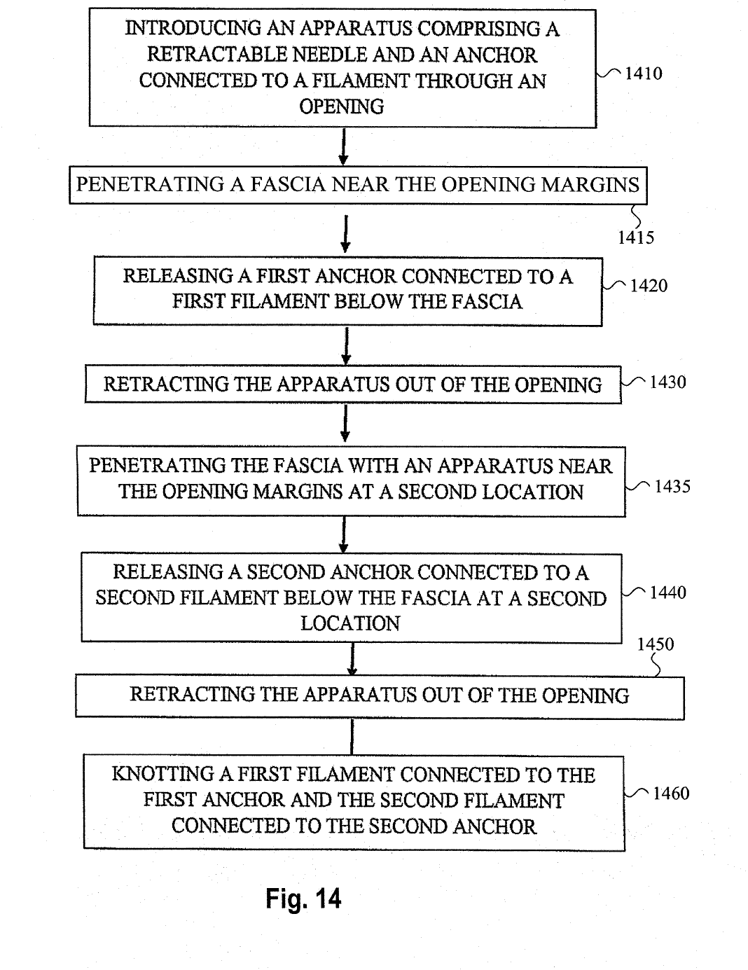

FIG. 14 shows a method of closing openings in an abdominal wall, according to exemplary embodiments of the disclosed subject matter.

Step 1410 discloses introducing an apparatus comprising a retractable needle and an anchor connected to a filament through an opening. The apparatus is inserted facing towards the abdominal cavity. Step 1415 discloses penetrating a fascia near the opening margins. Step 1420 discloses releasing a first anchor connected to a first filament below the fascia. Step 1430 discloses retracting the apparatus out of the opening. Step 1435 discloses penetrating the fascia with an apparatus near the opening margins at a second location. Step 1440 discloses releasing a second anchor connected to a second filament below the fascia at a second location. Step 1450 discloses retracting the apparatus out of the opening. Step 1460 discloses knotting a first filament connected to the first anchor and the second filament connected to the second anchor. The apparatus may be a retractable needle.

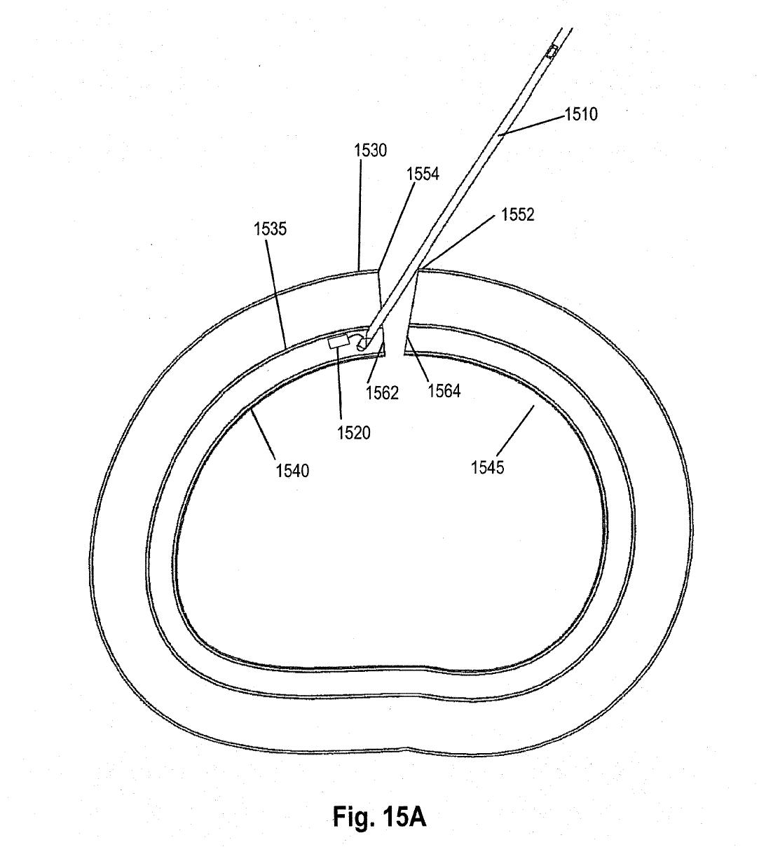

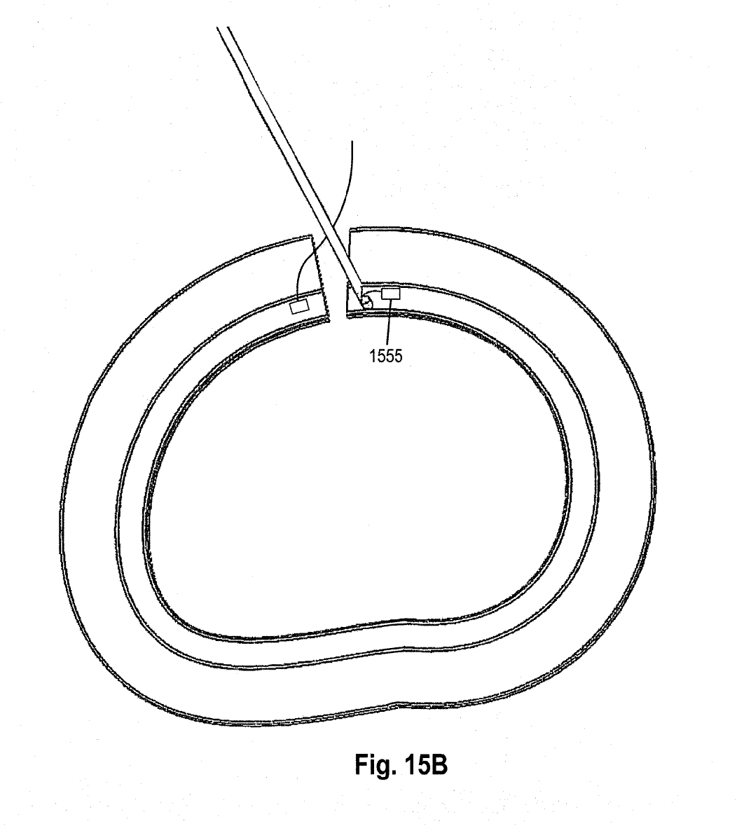

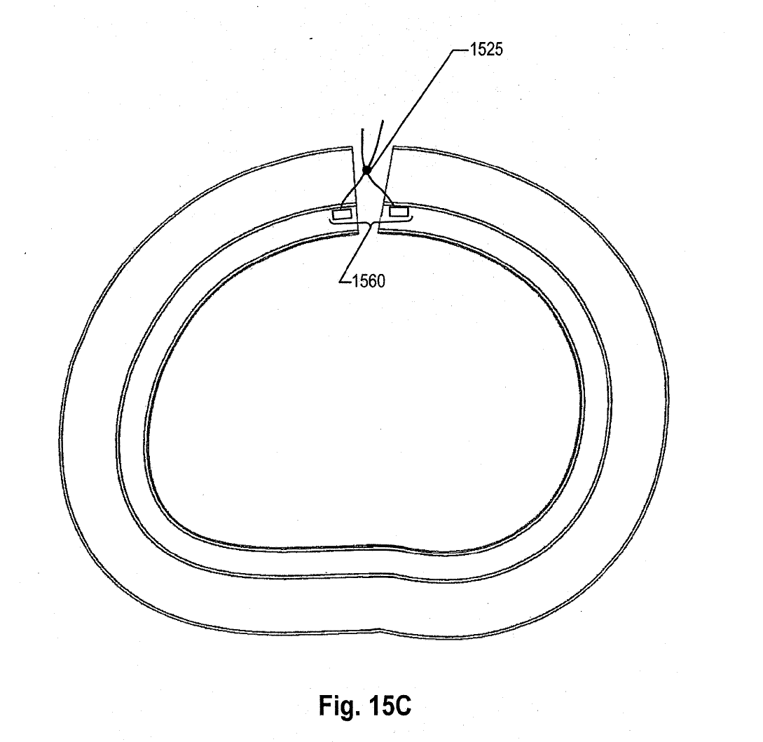

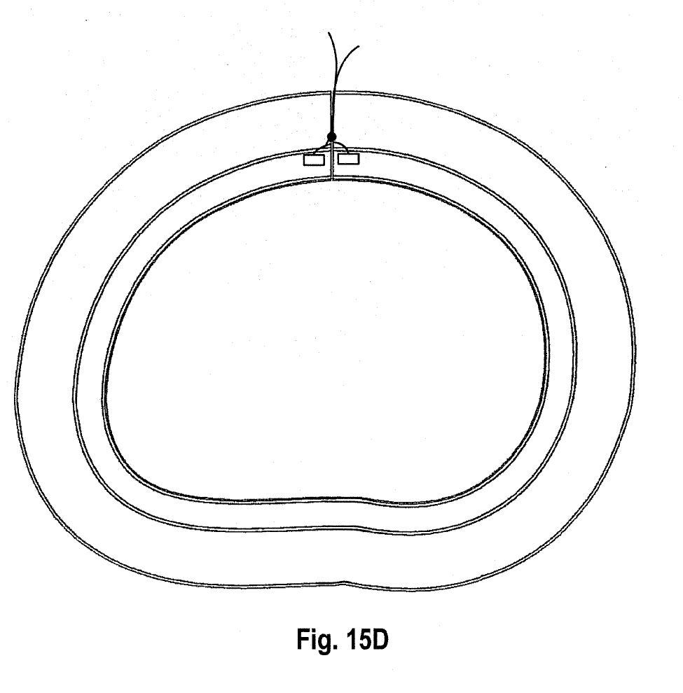

FIGS. 15A-15D show an apparatus for closing openings in an abdominal wall, according to exemplary embodiments of the subject matter. The apparatus 1510 is inserted into the patient's body facing towards the abdominal cavity 1545. The opening 1560 in the abdominal wall 1530 is defined by two margins 1552, 1554. After introducing the apparatus 1510 via the opening 1560, the apparatus 1510 penetrates the fascia 1535 and a first anchor 1520 is released below the fascia 1535. The first anchor 1520 is connected to a first filament 1562. Then, the apparatus 1510 is retracted from the opening 1560 and penetrates the fascia 1535 at a second location, at the other side of the opening 1560, where a second anchor 1555 is released. That is, each anchor is released at another side of the opening 1560, and each anchor is connected to a separate filament 1562, 1564. Then, the first filament 1562 and the second filament 1564 are knotted together, thereby bringing the first anchor 1520 and the second anchor 1555 in proximity and hence closing the opening.

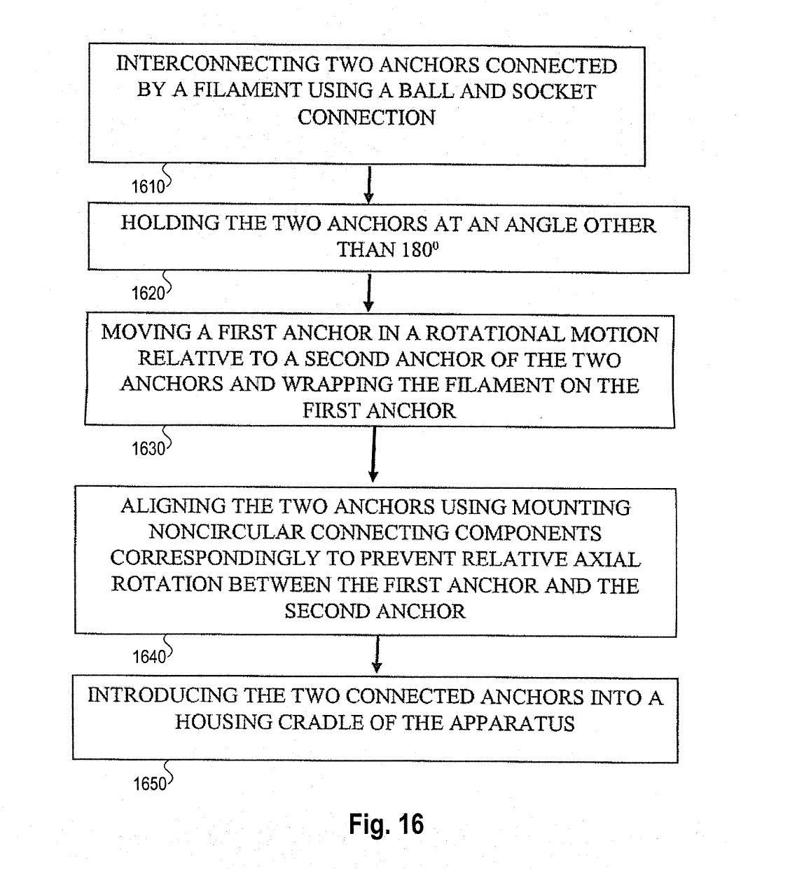

FIG. 16 shows a method of assembling an apparatus, according to exemplary embodiments of the disclosed subject matter. Step 1610 discloses interconnecting two anchors connected by a filament using a ball and socket connection. Step 1620 discloses holding the two anchors at an angle other than 180°. Step 1630 discloses moving a first anchor in a rotational motion relative to a second anchor of the two anchors and wrapping the filament on the first anchor. Step 1640 discloses aligning the two anchors using mounting noncircular connecting components correspondingly to prevent relative axial rotation between the first anchor and the second anchor. Step 1650 discloses introducing the two connected anchors into a housing cradle of the apparatus.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. An apparatus for fixating anchors to a body tissue, comprising:
a housing;
two or more interconnected anchors mounted inside the housing and configured to be reinforced to the body tissue;
wherein the housing has a first position in which the housing forms an aperture that enables release of an anchor of the two or more interconnected anchors and a second position preventing release of an anchor of the two or more interconnected anchors.

2. The apparatus of claim 1, wherein said housing comprises a retractable needle located in a distal end of the housing.

3. The apparatus of claim 2, wherein the needle protrudes from the two or more interconnected anchors when the housing is in the second position.

4. (canceled)

5. The apparatus of claim 2, wherein said housing has a third position in which the needle does not protrude from the two or more interconnected anchors while preventing release of an anchor of the two or more interconnected anchors.

6. The apparatus of claim 1, wherein the two or more interconnected anchors are connected via filaments.

7. The apparatus of claim 6, wherein at least a portion of the filaments are wrapped around the two or more interconnected anchors before released from the apparatus.

8. (canceled)

9. The apparatus of claim 6, wherein the two or more interconnected anchors have variable cross-sectional area, wherein the filaments are wrapped on a narrow portion of the two or more interconnected anchors.

10. The apparatus of claim 1, further comprises a cradle for storing the anchors, said cradle is located inside the housing and has a distal blunt tip.

11. The apparatus of claim 10, wherein an inner wall located at a distal end of the cradle forms a slope between the other anchors positioned in the cradle during release of an anchor from the aperture.

12. The apparatus of claim 1, further comprises a pusher for pushing the two or more interconnected anchors when releasing the anchor from the aperture.

13. The apparatus of claim 12, wherein the pusher comprises a ratchet mechanism.

14.-15. (canceled)

16. The apparatus of claim 2, wherein the needle is provided with a cutting element for cutting a filament connected to an anchor.

17. (canceled)

18. An apparatus, comprising:
a housing comprising a retractable needle located in a distal end of the housing;
two or more anchors mounted in a cradle having a blunt tip, said cradle is inside the housing;
wherein the blunt tip may be retracted and the sharp needle exposed during penetration through a more resilient tissue.

19. The apparatus of claim 18, wherein an anchor of the two or more anchors is released from the housing when the cradle protrudes from the needle.

20. The apparatus of claim 18, further comprises a spring connected to the cradle, wherein the spring is compressed and the cradle is retracted when the resilient tissue is hard, enabling the needle to protrude beyond the cradle.

21.-27. (canceled)

28. A method for attaching a mesh to a cavity wall, comprising:
introducing a mesh into the cavity;
penetrating with an apparatus comprising anchors through the cavity wall; approximating the mesh to a body tissue;
penetrating with the apparatus comprising anchors connected by filaments through the mesh at a first location;
penetrating with the apparatus comprising anchors through the body tissue; releasing a first anchor from the apparatus within the body tissue;
retracting the apparatus from the body tissue and from the mesh within the cavity wall;
penetrating with the apparatus comprising anchors connected by filaments through the mesh at a second location;
penetrating with the apparatus comprising anchors through the body tissue at a second location;
releasing a second anchor within the body tissue at the second location; retracting the apparatus from the body tissue and from the mesh within the cavity wall;
said second anchor being connected to the first anchor via a filament.

29. The method according to claim 28, further comprises inflating the cavity before penetrating to the cavity wall.

30. The method according to claim 28, wherein the apparatus is inserted percutaneously through the abdominal wall without the need for a trocar.

31. The method according to claim 28, wherein the apparatus comprises a retractable needle.

32. (canceled)

33. The method according to claim 28, wherein the body tissue is a fascia.

34. The method according to claim 28, further comprises cutting the filament.

35. The method according to claim 28, further comprises removing the apparatus from the cavity.

36.-38. (canceled)
